(19) United States
(12) Patent Application Publication
(10) Pub. No.: US 2023/0000884 A1
(43) Pub. Date: Jan. 5, 2023

(54) COMBINATION THERAPY FOR TREATING COVID-19

(71) Applicant: Luckwel Pharmaceuticals Inc., Cambridge, MA (US)

(72) Inventor: Kingrich Lee, Camrbidge, MA (US)

(21) Appl. No.: 17/364,331

(22) Filed: Jun. 30, 2021

(51) Int. Cl. A61K 31/665; A61K 31/404; A61K 31/357; A61K 45/06; A61P 11/08; A61P 31/06; A61P 31/14

(52) U.S. Cl. CPC A61K 31/665; A61K 31/404; A61K 31/357; A61K 45/06; A61P 11/08; A61P 31/06; A61P 31/14

(57) ABSTRACT

Methods for treating SARS-CoV-2 infection in a subject in need thereof are described. Methods for ameliorating symptoms of COVID-19 disease due to SARS-CoV-2 infection and are also described. The methods comprise administering to the subject an antiviral agent, a nucleoside reverse transcriptase inhibitor, an agent for treating tuberculosis, an agent for treating fever or systemic autoimmune disease with multisystem involvement, and an agent that acts as a bronchodilator or a bronchodilator and cough suppressant, expectorant combination.

### CROSS-REFERENCE TO RELATED APPLICATIONS

This field is intentionally left blank.

### TECHNICAL FIELD

The subject matter described herein generally relates to methods for treating coronavirus infections, particularly methods for treating coronavirus disease 2019 (COVID-19) caused by severe acute respiratory syndrome coronavirus 2 (SARS-CoV-2).

### BACKGROUND

SARS-CoV-2, which was previously named as 2019 novel coronavirus (2019-nCoV), belongs to the family of viruses known as coronaviruses. It is a positive-sense single-stranded RNA virus and contains four structural proteins, known as the S (spike), E (envelope), M (membrane), and N (nucleocapsid) proteins. The N protein contains the RNA genomic information. The S, E, and M proteins together create the viral envelope.

Similar to other viruses, SARS-CoV-2 causes infections by going through a viral replication cycle. The replication cycle begins with fusion of the virus with a host cell wherein the S protein is responsible for the initial attachment of the virus to the host cell. After entering into the host cell, the virus releases nucleic acid and forces the cell to replicate the viral genome. Transcription and translation subsequently occurs for protein synthesis and assembly of viral components. The newly formed virus is released from the host cell to the extracellular space. The viral load can cause pathogenesis after increasing to a certain point. The common symptoms of COVID-19 include fever, cough and shortness of breath.

COVID-19 appears to have a lower fatality/mortality rate than other coronavirus diseases, such as severe acute respiratory syndrome (SARS) or Middle East respiratory syndrome (MERS). However, COVID-19 appears to have a higher fatality/mortality rate than the seasonal flu. For example, particular studies have determined that the fatality/mortality rate associated with COVID-19 was higher than 2 percent. In any event, COVID-19 became a global pandemic in 2020 and could have severe impacts on global health and the economy.

Accordingly, a method that can inhibit the replication of severe acute respiratory syndrome coronavirus 2 (SARS-CoV-2) and relieve the symptoms of COVID-19 is desired for treating C OVID-19.

### BRIEF SUMMARY

The following aspects and embodiments thereof described and illustrated below are meant to be exemplary and illustrative, not limiting in scope.

In one aspect, the invention features a method of treating COVID-19 disease, or for ameliorating COVID-19 symptoms in a subject. The method involves administering to the subject an antiviral agent, a nucleoside reverse transcriptase inhibitor, and an agent for treating fever or systemic autoimmune disease with multisystem involvement. In some embodiments, the method further comprises administering a bronchodilator therapy comprising one or more of a bronchodilator, a cough suppressant, and an expectorant. In some embodiments, the method further comprises administering an agent for treating tuberculosis. The methods described treat both infection and symptoms caused by SARS-CoV-2 and SARS-CoV-2 variants.

In some embodiments, the antiviral agent, the nucleoside reverse transcriptase inhibitor, the agent for treating tuberculosis, the agent for treating fever or systemic autoimmune disease with multisystem involvement, and the bronchodilator are administered simultaneously. In some they are administered sequentially.

The method of any of claims 1-4, wherein the administering comprises administering 4-600 mg 1-3 times per day of each of the antiviral agent, nucleoside reverse transcriptase inhibitor, agent for treating tuberculosis, agent for treating fever or systemic autoimmune disease with multisystem involvement, and bronchodilator.

Antiviral agents compatible with the present methods include, but are not limited to, umifenovir oseltamivir, zanamivir, laninamivir, and peramivir, amantadine, rimantadine, and baloxavir, favipiravir, galidesivir, and remdesivir. In some embodiments, the antiviral agent for treating influenza comprises umifenovir.

Nucleoside reverse transcriptase inhibitors compatible with the present methods include, but are not limited to, didanosine, tenofovir, adefovir, marboxil abacavir (ABC), emtricitabine (FTC), lamivudine (3TC), stavudine (d4T), zidovudine (AZT, ZDV), evavirenz (EFV), nevirapine (NVP), dalevirdine (DLV), amprenavir, fosamprenavir (FPV), indinavir (IDV), lopinavir (LPV), nelfinavir (NFV), ritonavir (RTV), saqunavir (SQV), atazanavir (ATV), darunavir (DRV), and tipranavir (TPV). In some embodiments, the nucleoside reverse transcriptase inhibitor comprises fosamprenavir (FPV).

Agents for treating tuberculosis compatible with the present methods include, but are not limited to, rifampicin, isoniazid, ethambutol, pyrazinamide, and ethionamide. In some embodiments, the agent for treating tuberculosis comprises rifampicin and/or isoniazid.

Agents for treating fever or systemic autoimmune disease with multisystem involvement compatible with the present methods include, but are not limited to, chloroquine, hydroxychloroquine, and dihydroartemisinin (also known as artenimol), quinine, pramaquine, quinidine, artemether, artemisinin, artemotil, and artesunate. In some embodiments the agent for treating fever or systemic autoimmune disease with multisystem involvement comprises dihydroartemisinin (also known as artenimol).

Bronchodilators compatible with the present methods include, but are not limited to, albuterol (Salbutamol), levosalbutamol/levalbuterol, pirbuterol (Maxair), epinephrine (Primatene Mist), racemic epinephrine, ephedrine (Bronkaid), and terbutaline. In some embodiments, the bronchodilator comprises albuterol.

In another aspect, the invention features a pharmaceutical composition, comprising: a therapeutically effective amount of each of an antiviral agent, a nucleoside reverse transcriptase inhibitor, and an agent for treating fever or systemic autoimmune disease with multisystem involvement, wherein each therapeutically effective amount is in a unit dosage form comprising a pharmaceutically acceptable excipient. In some embodiments, the pharmaceutical composition further comprises a bronchodilator therapy comprising one or more of a bronchodilator, a cough suppressant, and an expectorant, wherein each therapeutically effective amount is in a unit dosage form comprising a pharmaceutically acceptable excipient. In some embodiments, the pharmaceutical composition further comprises an agent for treating tuberculosis, wherein each therapeutically effective amount is in a unit dosage form comprising a pharmaceutically acceptable excipient. The therapeutically effective amount for each of the antiviral agent, nucleoside reverse transcriptase inhibitor, agent for treating fever or systemic autoimmune disease with multisystem involvement, and bronchodilator therapy ranges from 5 to 600 mg.

Antiviral agents compatible with the present compositions include, but are not limited to, oseltamivir, zanamivir, laninamivir, peramivir, favipiravir, galidesivir, remdesivir, and fosamprenavir (FPV). In some embodiments, the antiviral agent comprises umifenovir.

Nucleoside reverse transcriptase inhibitors compatible with the present compositions include, but are not limited to, didanosine, tenofovir, adefovir, and fosamprenavir (FPV). In some embodiments, the nucleoside reverse transcriptase inhibitor comprises fosamprenavir (FPV).

Agents for treating tuberculosis compatible with the present compositions include, but are not limited to, rifampicin, isoniazid, ethambutol, pyrazinamide, and ethionamide. In some embodiments, the agent for treating tuberculosis comprises rifampicin and/or isoniazid and/or pyrazinamide.

Agents for treating fever or systemic autoimmune disease with multisystem involvement compatible with the present compositions include, but are not limited to, chloroquine, dihydroartemisinin (also known as artenimol), hydroxycholorquine, quinine, pramaquine, quinidine, artemether, artemisinin, artemotil, and artesunate. In some embodiments, the agent for treating fever or systemic autoimmune disease with multisystem involvement comprises dihydroartemisinin (also known as artenimol).

Bronchodilator therapies compatible with the present methods include, but are not limited to, albuterol (Salbutamol), levosalbutamol/levalbuterol, pirbuterol (Maxair), epinephrine (Primatene Mist), racemic epinephrine, ephedrine (Bronkaid), terbutaline, and Guaifenesin. In some embodiments, the bronchodilator therapy comprises albuterol, Guaifenesin, and dextromethorphan

In another aspect, the invention features a kit comprising three discrete compartments, wherein the compartments comprise, an antiviral agent, a nucleoside reverse transcriptase inhibitor, and an agent for treating fever systemic autoimmune disease with multisystem involvement respectively. In some embodiments, the kit further comprises a fourth discrete compartment comprising a bronchodilator therapy comprising one or more of a bronchodilator, a cough suppressant, and an expectorant. In some embodiments, the kit further comprises a fifth discrete compartment, wherein the compartment comprises, an agent for treating tuberculosis.

In some embodiments, the kit further comprises instructions for administering the antiviral agent, the nucleoside reverse transcriptase inhibitor, the agent for treating tuberculosis, the agent for treating fever or systemic autoimmune disease with multisystem involvement, and the bronchodilator therapy. In some embodiments, each of the antiviral agent, the nucleoside reverse transcriptase inhibitor, the agent for treating tuberculosis, the agent for treating fever or systemic autoimmune disease with multisystem involvement, and the bronchodilator is in an amount of 4-600 mg.

In some embodiments, the kit comprises a unit dose of each of an antiviral agent, a nucleoside reverse transcriptase inhibitor, an agent for treating tuberculosis, an agent for treating fever or systemic autoimmune disease with multisystem involvement, and a bronchodilator therapy comprising one or more of a bronchodilator, a cough suppressant, and an expectorant.

In addition to the exemplary aspects and embodiments described above, further aspects and embodiments will become apparent by reference to the examples and by study of the following descriptions.

Additional embodiments of the present methods and compositions, and the like, will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment herein. Additional aspects and advantages of the present compositions and methods are set forth in the following description and claims, particularly when considered in conjunction with the accompanying examples.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to an “excipient” includes a single excipient as well as two or more of the same or different excipients, and the like.

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value. In some embodiments, “about” means that the item, parameter or term so qualified encompasses a range of plus or minus ten percent above and/or below the value of the stated item, parameter or term.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“Therapeutically effective amount” as applied to the biologically active ingredient means that amount of the active ingredient which is generally sufficient to effect a desired change in the subject. For example, where the desired effect is a reduction in an autoimmune disorder symptom, an effective amount of the active ingredient is that amount which causes at least a substantial reduction of the autoimmune disorder symptom, and without resulting in significant toxicity.

“Subject” or “patient” means a human or non-human subject receiving medical or veterinary care. Accordingly, the method as disclosed herein can be used in treating any animal, such as, for example, mammals, or the like.

“Treating” means to alleviate (or to eliminate) at least one symptom of a condition or disorder, such as, for example, cough, fever or systemic autoimmune disease with multisystem involvement, shortness of breath, or the like, either temporarily or permanently.

### II. Combination Therapy

In one aspect, a method to treat COVID-19 in a subject in need thereof is provided. The method comprises administering to the subject a therapeutically effective amount of an antiviral agent, a nucleoside reverse transcriptase inhibitor, and an agent for treating fever or systemic autoimmune disease with multisystem involvement. It is generally contemplated that a therapeutically effective amount for each of the active agents would be from about 0.001 mg/kg to about 20 mg/kg body weight, such as from about 0.01 mg/kg to about 5 mg/kg body weight.

The antiviral agent, nucleoside reverse transcriptase inhibitor, and agent for treating fever or systemic autoimmune disease with multisystem involvement can be administered simultaneously, in a combination of simultaneous and sequential administration, or sequentially. Sequential administration refers to administration of one active agent within seconds, minutes, or hours of the immediately previous administration of another active agent. The order of the administration of the active agents is inconsequential.

In some embodiments, the method further comprises administering a bronchodilator therapy comprising one or more of a bronchodilator, a cough suppressant, and an expectorant to treat the symptoms caused by SARS-CoV-2. In some embodiments, the method further comprises administering an agent for treating tuberculosis.

In some embodiments, the administration comprises administering 4-300 mg/1-3 times/per day of each of the antiviral agent, nucleoside reverse transcriptase inhibitor, agent for treating fever or systemic autoimmune disease with multisystem involvement, agent for treating tuberculosis, and a bronchodilator therapy comprising one or more of a bronchodilator, a cough suppressant, and an expectorant.

In another aspect, a pharmaceutical composition is provided. The pharmaceutical composition comprising an antiviral agent, a nucleoside reverse transcriptase inhibitor, an agent for treating fever or systemic autoimmune disease with multisystem involvement, and a pharmaceutically acceptable excipient. In some embodiments, the pharmaceutical composition further comprises a bronchodilator therapy comprising one or more of a bronchodilator, a cough suppressant, and an expectorant. In some embodiments, the pharmaceutical composition further comprises an agent for treating tuberculosis.

The pharmaceutically acceptable excipient refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that the pharmaceutical composition can include one or more pharmaceutically acceptable excipients that facilitate processing of an active ingredient into pharmaceutically acceptable compositions. Non-limiting examples of pharmaceutically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003), each of which is hereby incorporated by reference in its entirety.

In another aspect, a kit is provided. In some embodiments, the kit comprises three compartments for containing the antiviral agent, the nucleoside reverse transcriptase inhibitor, and an agent for treating fever or systemic autoimmune disease with multisystem involvement, respectively. In some embodiments, the kit comprises four compartments for containing the antiviral agent, the nucleoside reverse transcriptase inhibitor, an agent for treating fever or systemic autoimmune disease with multisystem involvement, and a bronchodilator therapy comprising one or more of a bronchodilator, a cough suppressant, and an expectorant, respectively. In some embodiments, the kit comprises five compartments for containing the antiviral agent, the nucleoside reverse transcriptase inhibitor, an agent for treating fever or systemic autoimmune disease with multisystem involvement, a bronchodilator therapy comprising one or more of a bronchodilator, a cough suppressant, and an expectorant, and an agent for treating tuberculosis, respectively. In some embodiments, the kit comprises one compartment comprising the pharmaceutically acceptable composition comprising the antiviral agent for treating influenza, the nucleoside reverse transcriptase inhibitor, an agent for treating fever, and a pharmaceutically acceptable excipient. In some embodiments, the kit comprises one compartment comprising the pharmaceutically acceptable composition comprising the antiviral agent for treating influenza, the nucleoside reverse transcriptase inhibitor, an agent for treating fever or systemic autoimmune disease with multisystem involvement, a bronchodilator therapy comprising one or more of a bronchodilator, a cough suppressant, and an expectorant, and a pharmaceutically acceptable excipient. In some embodiments, the kit comprises one compartment comprising the pharmaceutically acceptable composition comprising the antiviral agent for treating influenza, the nucleoside reverse transcriptase inhibitor, an agent for treating fever or systemic autoimmune disease with multisystem involvement, a bronchodilator therapy comprising one or more of a bronchodilator, a cough suppressant, and an expectorant, an agent for treating tuberculosis, and a pharmaceutically acceptable excipient. The kit can further comprise instructions for administration of the aforementioned active agents.

As used herein, the antiviral agent, the nucleoside reverse transcriptase inhibitor, the agent for treating tuberculosis, the agent for treating fever or systemic autoimmune disease with multisystem involvement, and the bronchodilator refer to either the active ingredient or the commercial product comprising the ingredient.

### Antiviral Agent for Treating Influenza

Most antiviral agents act by inhibiting replication of the virus. They target key enzyme required for viral replication cycle. The antiviral agent disclosed herein for treatment of infection due to SARS-CoV-2 or for treatment of symptoms associated with SARS-CoV-2 infection is, in one embodiment, a neuraminidase inhibitor, which is a competitive inhibitor of influenza's neuraminidase enzyme. The enzyme cleaves the sialic acid which is found on glycoproteins on the surface of human cells that helps new virions to exit the cell. Thus, the antiviral agent disclosed herein prevents or inhibits new viral particles from being released. The antiviral agent disclosed herein can also be those inhibiting the fusion between the viral envelope and the cell membrane of the target cells. Due to the similarity between the symptoms of influenza and COVID-19 and the common replication cycle, it is contemplated that an antiviral agent for treating influenza may also prevent or inhibit SARS-CoV-2 virus from being replicated to some extent and/or alleviate the symptoms of COVID-19.

The antiviral agents contemplated for use in the methods and kits disclosed herein include oseltamivir (Tamiflu), zanamivir (Relenza), laninamivir (Inavir), and peramivir (Rapivab), amantadine, rimantadine, umifenovir, baloxavir marboxil, favipiravir, galidesivir, and remdesivir. The exemplary antiviral agents disclosed herein are generally approved by a government regulation agency. For example, both oseltamivir and zanamivir were approved in the US and Europe for treatment and prevention of influenza A and B. Peramivir (Rapivab) was approved in the US for treating influenza infection in adults. The antiviral agent can be in any form as approved by a government regulation agency, such as the US FDA. The form can be tablets or capsules.

The recommended dose for the exemplary antiviral agents can be found in their respective labels. For example, the recommended dose for umifenovir is 100 mg by oral once or twice daily. The recommended dose for zanamivir is 10 mg by oral inhalation once or twice daily. The recommended dose for peramivir is 600 mg intravenous infusion for a minimum of 15 minutes per day.

In some embodiments, the antiviral agent disclosed herein is umifenovir. In some embodiments, the effective amount of oseltamivir that can be administered is the same as the dose approved by the US FDA, for example, 100 mg oral once or twice daily.

In some embodiments, the antiviral agent disclosed herein is zanamivir. In some embodiments, the effective amount of zanamivir that can be administered is the same as the dose approved by the US FDA, for example, 10 mg oral inhalation once or twice daily.

In some embodiments, the antiviral agent disclosed herein is peramivir. In some embodiments, the effective amount of peramivir that can be administered is the same as the dose approved by the US FDA, for example, 600 mg or 200 mg by intravenous infusion for a minimum of 15 minutes per day.

In some embodiments, the antiviral agent disclosed herein is amantadine

In some embodiments, the antiviral agent disclosed herein is rimantadine,

In some embodiments, the antiviral agent disclosed herein is baloxavir marboxil.

In some embodiments, the antiviral agent disclosed herein is favipiravir,

In some embodiments, the antiviral agent disclosed herein is galidesivir,

In some embodiments, the antiviral agent disclosed herein is remdesivir

### Nucleoside Reverse Transcriptase Inhibitor

The nucleoside reverse transcriptase inhibitor refers to the analogs of cytidine, guanosine, thymidine and adenosine, which have been approved by the US FDA to treat chronic hepatitis B and/or to prevent and treat HIV/AIDS. The analogs can serve as poison building blocks (chain terminators) for both viral and host DNA, disrupting the replication of virus and causing respectively the desired antiviral effect and drug toxicity/side effects.

In some embodiments, the nucleoside reverse transcriptase inhibitor is selected from didanosine (ddI), tenofovir (TDF), adefovir (ADV), abacavir (ABC), emtricitabine (FTC), lamivudine (3TC), stavudine (d4T), zidovudine (AZT, ZDV), evavirenz (EFV), nevirapine (NVP), dalevirdine (DLV), amprenavir, fosamprenavir (FPV), indinavir (IDV), lopinavir (LPV), nelfinavir (NFV), ritonavir (RTV), saqunavir (SQV), atazanavir (ATV), darunavir (DRV), and tipranavir (TPV). The nucleoside reverse transcriptase inhibitor can be in any form as approved by a government regulation agency, such as the US FDA. The form can be tablets or capsules. The recommended dose for the exemplary nucleoside reverse transcriptase inhibitors can be found in their respective labels.

In some embodiments, the nucleoside reverse transcriptase inhibitor is didanosine. The effective amount of didanosine administered can be the same as the dosage approved by the US FDA, for example, 400 mg once daily or 200 mg twice daily.

In some embodiments, the nucleoside reverse transcriptase inhibitor is tenofovir. The effective amount of tenofovir administered can be the same as approved by the US FDA, for example, 300 mg tablet once daily taken orally.

In some embodiments, the nucleoside reverse transcriptase inhibitor is adefovir. The effective amount of adefovir administered can be the same as the dosage approved by the US FDA, for example, 10 mg adefovir once daily, once every two days, or once every three days orally depending on the creatinine clearance.

### Agent for Treating Tuberculosis

SARS-CoV-2 viruses appear to have a transmission similar to, for example, tuberculosis transmission. When people with active pulmonary tuberculosis cough, sneeze, speak, sing, or spit, they expel infectious aerosol droplets of about 0.5 to 5.0 μm in diameter. A single sneeze can release up to 40,000 droplets. Each of these droplets may transmit the disease, since the infectious dose of tuberculosis is very small (the inhalation of fewer than 10 bacteria may cause an infection).

People with prolonged, frequent, or close contact with individuals having tuberculosis are at particularly high risk of becoming infected, with an estimated 22% infection rate. A person with active, but untreated, tuberculosis may infect 10-15 (or more) other people per year. Transmission is only expected to occur from individuals with active tuberculosis—those with latent infection are not thought to be contagious. The probability of transmission from one person to another depends upon several factors, including the number of infectious droplets expelled by the carrier, the effectiveness of ventilation the duration of exposure, the virulence of the M. tuberculosis strain, the level of immunity in the uninfected person, and others. The cascade of person-to-person spread can be circumvented by segregating those with active (“overt”) infection and providing them anti-tuberculosis drug regimens. After about two weeks of effective treatment, subjects with nonresistant active infections generally do not remain contagious to others. If someone does become infected, it typically takes three to four weeks before the newly infected person becomes infectious enough to transmit the disease to others.

Thus, administration of an anti-tuberculosis agent to a subject having infection or symptoms due to SARS-CoV-2, such as a pharmaceutical used for the treatment of tuberculosis, is contemplated to prevent or inhibit COVID-19 transmission between subjects. The anti-tuberculosis agent can be rifampicin, isoniazid, ethambutol, pyrazinamide, and ethionamide. The anti-tuberculosis agent can be in any form as approved by a government regulation agency, such as the US FDA. The form can be tablets or capsules. The recommended dose for the exemplary anti-tuberculosis agents can be found in their respective labels. In some embodiments, the anti-tuberculosis comprises rifampicin and/or isoniazid.

The effective amount of rifampicin and isoniazid can be the same as the dosage approved by the US FDA. Adults and older children-600 mg of rifampin and 300 mg of isoniazid once a day orally; in one embodiment, a dose maximum of about 300 mg rifampin and 150 mg isoniazid once daily is contemplated.

Agent for Treating Fever or Systemic Autoimmune Disease with Multisystem Involvement

The anti-fever agent can be any anti-fever agent that can treat fever. Chloroquine and dihydroartemisinin (also known as artenimol), hydroxycholorquine, and quinine, pramaquine, quinidine, artemether, artemisinin, artemotil, and artesunate are examples of anti-fever agents contemplated for treatment of fever in subjects with COVID-19. The anti-fever agent can be in any form as approved by a government regulation agency, such as the US FDA. The form can be tablets or capsules. The recommended dose for the exemplary anti-fever agents can be found in their respective labels.

The effective amount of chloroquine can be the same dosage as approved by the US FDA, for example, 155 mg daily taken orally, 310 mg once weekly, or 5 mg/kg. The effective amount of dihydroartemisinin (also known as artenimol) can be the same dosage as approved by the China FDA or World Health Organization-Geneva, for example, 120 mg initially, then 60 mg daily for a further 4 to 6 days.

### Bronchodilator

It has been found that the lungs of patients with infection due to SARS-CoV-2 are filled with jelly-like secretions caused by the virus, resulting in the loss of the lung's ventilation function and shortness of breath. The bronchodilator disclosed herein refers to an active agent that dilate the bronchi and bronchioles. The bronchodilator in general is a β₂-adrenergic agonist, which can relax the bronchial smooth muscle and relieve bronchial smooth muscle spasm. The bronchodilator therefore can reduce resistance in the respiratory airway and increase airflow to the lungs, thereby relieving the shortness of breath.

Exemplary bronchodilator includes albuterol (Salbutamol), levosalbutamol/levalbuterol, pirbuterol (Maxair), epinephrine (Primatene Mist), racemic epinephrine, ephedrine (Bronkaid), and terbutaline. The effective amount for each of the bronchodilators can be the same as approved by the US FDA. The bronchodilator can be in any form as approved by a government regulation agency, such as the US FDA. The form can be tablets or capsules. The recommended dose for the exemplary bronchodilator can be found in their respective labels.

In some embodiments, the bronchodilator is albuterol in the form of a tablet. In some embodiments, the effective amount of albuterol can be around 12-16 mg per day. In other embodiments, the bronchodilator is albuterol in the form of a liquid that is nebulized into the airway.

In another aspect, a pharmaceutical composition is provided. The pharmaceutical composition comprising an antiviral agent, a nucleoside reverse transcriptase inhibitor, an agent for treating fever, and a pharmaceutically acceptable excipient. In some embodiments, the pharmaceutical composition further comprises a bronchodilator therapy comprising one or more of a bronchodilator, a cough suppressant, and an expectorant. In some embodiments, the pharmaceutical composition further comprises an agent for treating tuberculosis. In one embodiment, each active agent is a discrete unit dosage.

In another aspect, a kit is provided. The kit comprises three compartments for containing the antiviral agent, the nucleoside reverse transcriptase inhibitor, and the agent for treating fever, respectively. In some embodiments, the kit comprises four compartments. In some embodiments the kit comprises five compartments. In some embodiments, the kit further comprises a bronchodilator therapy comprising one or more of a bronchodilator, a cough suppressant, and an expectorant. In some embodiments, the kit further comprises an agent for treating tuberculosis. The kit can further comprise instructions for administration of the aforementioned active agents.

In Example 1, a treatment regime is described and contemplated for treating infection and/or the symptoms caused by COVID-19 in a patient who unfortunately developed cough, fever or systemic autoimmune disease with multisystem involvement, and shortness of breath. In the method, Fosamprenavir, Umifenovir, rifampicin and/or isoniazid, dihydroartemisinin (also known as artenimol), and albuterol are administered to the patient sequentially. That is, the patient takes each active agent within a defined period of time of about 5 minutes, 10 minutes, 30 minutes, 1 hour, 2 hours, 4 hours, or 8 hours. The dose for each active agent is not more than that approved by the US FDA for each of their targeted uses. The patient is expected to have cough, fever, and/or shortness of breath alleviated or reduced after one to two weeks of daily treatment.

### III. EXAMPLES

The following examples are illustrative in nature and are in no way intended to be limiting.

### Example 1

### Treatment of Covid-19

A patient diagnosed with infection by SARS-CoV-2 and is experiencing COVID-19 symptoms, such as cough, fever or systemic autoimmune disease with multisystem involvement, and shortness of breath can be treated with a combination therapy comprising Fosamprenavir, Umifenovir, and Artenimol. The effective amounts of each of Fosamprenavir, Umifenovir, and Artenimol can be around 50-600 mg per day.

### Example 2

### Treatment of Covid-19 Variant

A patient diagnosed with infection by a SARS-CoV-2 variant and is experiencing COVID-19 symptoms, such as cough, fever or systemic autoimmune disease with multisystem involvement, and shortness of breath, difficulty breathing, and mucus in the lungs can be treated with a combination therapy comprising Fosampreanvir, Umifenovir, and Artenimol, and a combination therapy comprising Albuterol, and Guaifenesin tablets.

### Example 3

### Treatment of Covid-19 in Tuberculosis Afflicted Patients

Patients with Tuberculosis (TB) disease diagnosed with infection by SARS-CoV-2 can be treated with a combination therapy comprising Fosamprenavir, Umifenovir, and Artenimol, and combination therapies comprising rifampicin and/or isoniazid and/or pyrazinamide, and albuterol and Guaifenesin tablets. The dose for each ingredient is approved by a doctor and is not more than that approved by the US FDA for each of their targeted uses. The patient experiences no cough, fever, and shortness of breath after administering the combination therapy comprising Fosamprenavir, Umifenovir, and Artenimol, rifampicin and/or isoniazid and/or pyrazinamide, and albuterol-Guaifenesin-dextromethorphan combination therapy for one to two weeks.

### Example 4

### Treatment of a Covid-19 Variant in Tuberculosis Afflicted Patients

Patients with Tuberculosis (TB) disease diagnosed with infection by a SARS-CoV-2 variant can be treated with a combination therapy comprising Fosamprenavir, Umifenovir, and Artenimol, and combination therapies comprising rifampicin and/or isoniazid and/or pyrazinamide, and albuterol-Guaifenesin-dextromethorphan combination therapy. The dose for each ingredient is approved by a doctor and is not more than that approved by the US FDA for each of their targeted uses. The patient experiences no cough, fever, and shortness of breath after administering the combination therapy comprising Fosamprenavir, Umifenovir, and Artenimol, rifampicin and/or isoniazid and/or pyrazinamide, and albuterol and Guaifenesin Kit tablets for one to two weeks.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method of treating COVID-19 disease, or for ameliorating COVID-19 symptoms in a subject in need thereof, comprising: administering to the subject an antiviral agent, a nucleoside reverse transcriptase inhibitor, and an agent for treating fever or systemic autoimmune disease with multisystem involvement.

2. The method of claim 1, further comprising administering a bronchodilator therapy comprising one or more of a bronchodilator, a cough suppressant, and an expectorant, wherein said administering treats infection and symptoms caused by SARS-CoV-2.

3. The method of claim 2, further comprising administering an agent for treating tuberculosis.

4-5. (canceled)

6. The method of claim 3, wherein the administering comprises administering 4-600 mg 1-3 times per day of each of the antiviral agent, nucleoside reverse transcriptase inhibitor, agent for treating tuberculosis, agent for treating fever or systemic autoimmune disease with multisystem involvement, and bronchodilator therapy.

7. The method of claim 1, wherein the antiviral agent is selected from umifenovir oseltamivir, zanamivir, laninamivir, and peramivir, amantadine, rimantadine, and baloxavir, favipiravir, galidesivir, and remdesivir; wherein the nucleoside reverse transcriptase inhibitor is selected from didanosine, tenofovir, adefovir, marboxil abacavir (ABC), emtricitabine (FTC), lamivudine (3TC), stavudine (d4T), zidovudine (AZT, ZDV), evavirenz (EFV), nevirapine (NVP), dalevirdine (DLV), amprenavir, fosamprenavir (FPV), indinavir (IDV), lopinavir (LPV), nelfinavir (NFV), ritonavir (RTV), saqunavir (SQV), atazanavir (ATV), darunavir (DRV), and tipranavir (TPV); and wherein the agent for treating fever or systemic autoimmune disease with multisystem involvement is selected from chloroquine, hydroxychloroquine, and dihydroartemisinin (also known as artenimol), quinine, pramaquine, quinidine, artemether, artemisinin, artemotil, and artesunate.

8-9. (canceled)

10. The method of claim 3, wherein the agent for treating tuberculosis is selected from rifampicin, isoniazid, ethambutol, pyrazinamide, and ethionamide.

11-13. (canceled)

14. The method of any of claim 2, wherein the bronchodilator is selected from albuterol (Salbutamol), levosalbutamol/levalbuterol, pirbuterol (Maxair), epinephrine (Primatene Mist), racemic epinephrine, ephedrine (Bronkaid), and terbutaline.

15. (canceled)

16. A pharmaceutical composition, comprising: a therapeutically effective amount of each of an antiviral agent, a nucleoside reverse transcriptase inhibitor, and an agent for treating fever or systemic autoimmune disease with multisystem involvement, wherein each therapeutically effective amount is in a unit dosage form comprising a pharmaceutically acceptable excipient.

17. The pharmaceutical composition of claim 16, further comprising a bronchodilator therapy comprising one or more of a bronchodilator, a cough suppressant, and an expectorant, wherein each therapeutically effective amount is in a unit dosage form comprising a pharmaceutically acceptable excipient.

18. The pharmaceutical composition of claim 17, further comprising an agent for treating tuberculosis, wherein each therapeutically effective amount is in a unit dosage form comprising a pharmaceutically acceptable excipient.

19. The pharmaceutical composition of claim 17, wherein the therapeutically effective amount for each of the antiviral agent, nucleoside reverse transcriptase inhibitor, agent for treating fever or systemic autoimmune disease with multisystem involvement, and bronchodilator therapy ranges from 5 to 600 mg.

20. The pharmaceutical composition of claim 16, wherein the antiviral agent is selected from oseltamivir, zanamivir, laninamivir, peramivir, favipiravir, galidesivir, remdesivir, and fosamprenavir (FPV); wherein the nucleoside reverse transcriptase inhibitor is selected from didanosine, tenofovir, adefovir, and fosamprenavir (FPV).

21-23. (canceled)

24. The pharmaceutical composition of claim 18, wherein the agent for treating tuberculosis is selected from rifampicin, isoniazid, ethambutol, pyrazinamide, and ethionamide.

25. (canceled)

26. The pharmaceutical composition of claim 16, wherein the agent for treating fever or systemic autoimmune disease with multisystem involvement is selected from chloroquine, dihydroartemisinin (also known as artenimol), hydroxycholorquine, quinine, pramaquine, quinidine, artemether, artemisinin, artemotil, and artesunate.

27. (canceled)

28. The pharmaceutical composition of claim 17, wherein the bronchodilator therapy is selected from albuterol (Salbutamol), levosalbutamol/levalbuterol, pirbuterol (Maxair), epinephrine (Primatene Mist), racemic epinephrine, ephedrine (Bronkaid), terbutaline, and Guaifenesin.

29. (canceled)

30. A kit comprising three discrete compartments, wherein the compartments comprise, an antiviral agent, a nucleoside reverse transcriptase inhibitor, and an agent for treating fever systemic autoimmune disease with multisystem involvement respectively.

31. The kit of claim 30, further comprising a fourth discrete compartment, wherein the compartment comprises, a bronchodilator therapy comprising one or more of a bronchodilator, a cough suppressant, and an expectorant.

32. The kit of claim 30, further comprising a fifth discrete compartment, wherein the compartment comprises, an agent for treating tuberculosis.

33. The kit of claim 32, further comprising instructions for administering the antiviral agent, the nucleoside reverse transcriptase inhibitor, the agent for treating tuberculosis, the agent for treating fever or systemic autoimmune disease with multisystem involvement, and the bronchodilator therapy.

34. The kit of claim 32, wherein each of the antiviral agent, the nucleoside reverse transcriptase inhibitor, the agent for treating tuberculosis, the agent for treating fever or systemic autoimmune disease with multisystem involvement, and the bronchodilator is in an amount of 4-600 mg.

35. (canceled)
